(19) United States
(12) Patent Application Publication
(10) Pub. No.: US 2024/0350805 A1
(43) Pub. Date: Oct. 24, 2024

(54) SYSTEMS AND METHODS OF IMPROVING SLEEP DISORDERED BREATHING

(71) Applicant: VANDERBILT UNIVERSITY, Nashville, TN (US)

(72) Inventor: David T. Kent, Nashville, TN (US)

(21) Appl. No.: 18/684,796

(22) PCT Filed: Aug. 25, 2022
(86) PCT No.: PCT/US2022/041575

§ 371 Date: Feb. 19, 2024

Related U.S. Application Data
(60) Provisional application No. 63/236,774, filed on Aug. 25, 2021.

(51) Int. Cl. A61N 1/36

(52) U.S. Cl. CPC A61N 1/3611; A61N 1/3601; A61N 1/36139; A61N 1/36175

(57) ABSTRACT

The present disclosure relates to systems and methods for improving sleep disordered breathing by stimulating the ansa cervicalis and activating the sternothyroid muscle and stimulating the phrenic nerve to activate the diaphragm.

### CROSS-REFERENCE TO RELATED APPLICATIONS

This application claims the benefit of U.S. Provisional Application No. 63/236,774, entitled “SYSTEMS AND METHODS OF IMPROVING SLEEP DISORDERED BREATHING,” filed 25 Aug. 2021. The entirety of this provisional application is hereby incorporated by reference for all purposes.

### TECHNICAL FIELD

The present disclosure relates to systems and methods of improving sleep disordered breathing via neuromodulation.

### BACKGROUND

Sleep disordered breathing (SDB) occurs when there is a partial or complete cessation of breathing that occurs many times throughout the night. Obstructive sleep apnea (OSA) is a type of SDB that involves cessation or significant decrease in airflow in the presence of breathing effort. It is the most common type of SDB and is characterized by recurrent episodes of upper airway collapse during sleep inducing repetitive pauses in breathing followed by reductions in blood oxygen saturation or neurologic arousal. The pathophysiology of OSA can involve factors such as craniofacial anatomy, airway collapsibility, and neuromuscular control of the upper airway dilator musculature. Electromyogram studies have shown that the tonic and phasic activity of the pharyngeal airway dilatory muscles (such as the genioglossus muscle) is progressively reduced from wakefulness to non-rapid eye movement to rapid eye movement.

Continuous positive airway pressure (CPAP) therapy is the frontline treatment for OSA. CPAP therapy utilizes machines, generally including a flow generator, tubing, and a mask designed to deliver a constant flow of air pressure to keep the airways continuously open in patients with OSA. However, the success of CPAP therapy is limited by compliance with reported rates ranging from 50% to 70%. Hypoglossal nerve stimulation (HNS) has now been established as an effective form of therapy for patients with obstructive sleep apnea (OSA) who are unable to tolerate positive airway pressure. This therapy works by protruding and stiffening the tongue muscle thereby dilating the pharyngeal airway. However, only a small subset of patients with OSA have anatomy suitable for hypoglossal nerve stimulation therapy, as many patients continue to suffer from airway collapse even with stimulation of hypoglossal nerve musculature.

### SUMMARY

The present disclosure relates to systems and methods for improving SDB. In an aspect, a therapy delivery system for improving SDB is provided. Such a system comprises a first electrode configured to deliver a first electrical signal to a target site proximate to the ansa cervicalis to stimulate the ansa cervicalis and activate the sternothyroid muscle and a second electrode configured to deliver a second electrical signal to target site proximate to the phrenic nerve to stimulate the phrenic nerve and activate the diaphragm. The system also includes a controller in electrical communication with the first electrode, the second electrode, and the power source and programmed to direct delivery of the first electrical signal to the target site to stimulate the ansa cervicalis and activate the sternothyroid muscle and programmed to direct delivery of the second electrical signal to the phrenic nerve to stimulate the phrenic nerve and activate the diaphragm to improve the sleep disordered breathing.

In another aspect, a method of improving SDB in a patient suffering therefrom is provided comprising delivering a first electrical signal to a target site proximate to an ansa cervicalis innervating the sternothyroid muscle and activating the sternothyroid muscle. The method further comprises delivering a second electrical signal to a phrenic nerve innervating the diaphragm activating the diaphragm. The method further includes improving the patient's sleep disordered breathing via delivery of the first and the electrical signals.

### BRIEF DESCRIPTION OF THE DRAWINGS

FIG. 1 is a flow chart depicting illustrative steps of a method of improving SDB in a patient suffering therefrom.

FIG. 2 is a schematic illustration of exemplary target sites for neuromodulation according to an aspect of the present disclosure.

FIG. 3 is a schematic illustration of exemplary target sites for neuromodulation according to an aspect of the present disclosure.

FIG. 4 is a schematic illustration of exemplary target sites for neuromodulation according to an aspect of the present disclosure.

FIG. 5 is a schematic illustration of an exemplary target site for neuromodulation according to an aspect of the present disclosure.

FIG. 6 is a block diagram depicting illustrative components of a neuromodulation system according to an aspect of the present disclosure.

FIG. 7 is a block diagram depicting illustrative components of a neuromodulation system according to an aspect of the present disclosure.

FIG. 8 is block diagram depicting illustrative components of a neurostimulator according to an aspect of the present disclosure.

### DETAILED DESCRIPTION

The present disclosure relates to systems and methods for improving SDB. Non-limiting examples of SDBs are increased upper airway resistance including snoring; upper airway resistance syndrome (UARS); and sleep apnea. Sleep apnea can include OSA, central sleep apnea (CSA), and mixed sleep apnea. Reference to “improving” a patient's SDB includes treating, reducing the symptoms of, mitigating, or preventing the SDB. In certain aspects, a method of improving a patient's SDB is preventative as opposed to reactionary in nature. In other words, a method of improving a patient's SDB according to certain aspects involves preventing SDB as opposed to detecting an apnea or hypopnea event, for example, and responding to such detected event. By preventing SDB, a treatment method can reduce the potential for airway collapse as opposed to reacting to a documented event. A patient suffering from SDB includes a mammal, such as a human being. As used herein with respect to a described element, the terms “a,” “an,” and “the” include at least one or more of the described element unless otherwise indicated. Further, the terms “or” and “and” refer to “and/or” and combinations thereof unless otherwise indicated. The terms “first,” “second,” etc. are not used in a quantitative sense unless indicated otherwise. In particular, a “first,” “second,” “third,” “fourth,” “fifth,” “sixth,” “seventh” electrode or electrical signal can be same electrode or the same electrical signal or can be different electrodes or different electrodes. As such, an electrode or electrical signal can be used to stimulate multiple target sites or an electrode or an electrical signal can be used to stimulate a single target site. As used herein a “patient” includes a mammal such as a human being. All devices as described herein are used for medical purposes and are therefore sterile.

The present disclosure provides methods and systems for treating SDB in a patient suffering therefrom by activating one or more infrahyoid strap muscles as well as the diaphragm. Activation of one or more infrahyoid muscles can be accomplished by stimulating an ansa cervicalis, including one or both of the superior root and the inferior root of the ansa cervicalis. Without wishing to be bound by a particular mechanism of action, it is believed that activation of infrahyoid muscles (e.g. tightening of these muscles) can reduce upper airway compliance (e.g. stiffen the upper airway). Upper airway compliance can indicate the potential of the airway to collapse and can be relevant to treating SDB. As explained below, the infrahyoid muscles include the sternohyoid muscle, the sternothyroid muscle, the omohyoid muscle, and the thyrohyoid muscle. Activation of the diaphragm can be accomplished by stimulating the phrenic nerve.

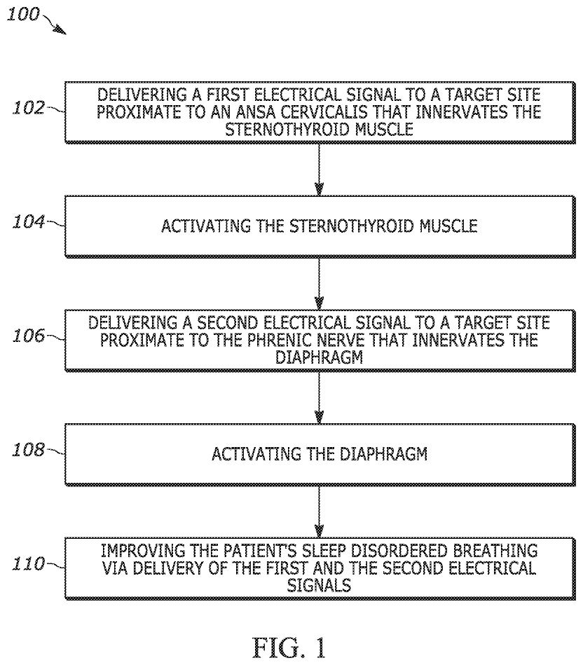

Referring to FIG. 1, in an aspect, a method 100 of treating SDB in a patient suffering therefrom comprises delivering a first electrical signal to a target site proximate to an ansa cervicalis that innervates at least the sternothyroid muscle 102. A target site is proximate to the ansa cervicalis such that delivering a neuromodulation signal activates the motor fibers of the ansa cervicalis to activate at least the sternothyroid muscle. Method 100 further includes activating the sternothyroid muscle 104. Method 100 further includes delivering a second electrical signal to a target site proximate to the phrenic nerve 106 that innervates the diaphragm. A target site is proximate to the phrenic nerve such that delivering an electrical signal activates the phrenic nerve to activate the diaphragm. Method 100 further includes activating the diaphragm 108. Method 100 further comprises improving the patient's SBD via delivery of the first electrical signal and the second electrical signal 110.

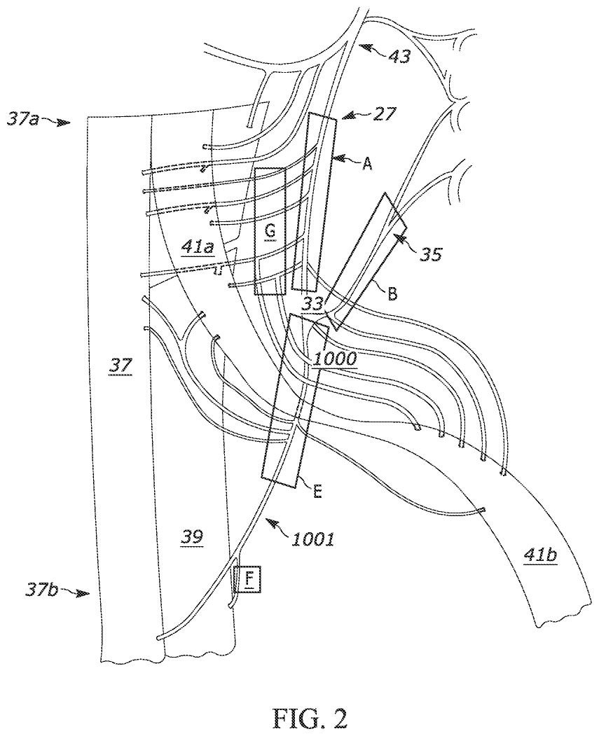

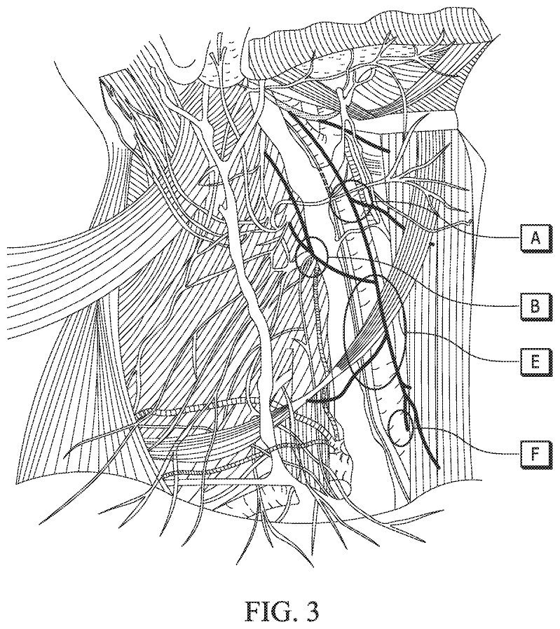

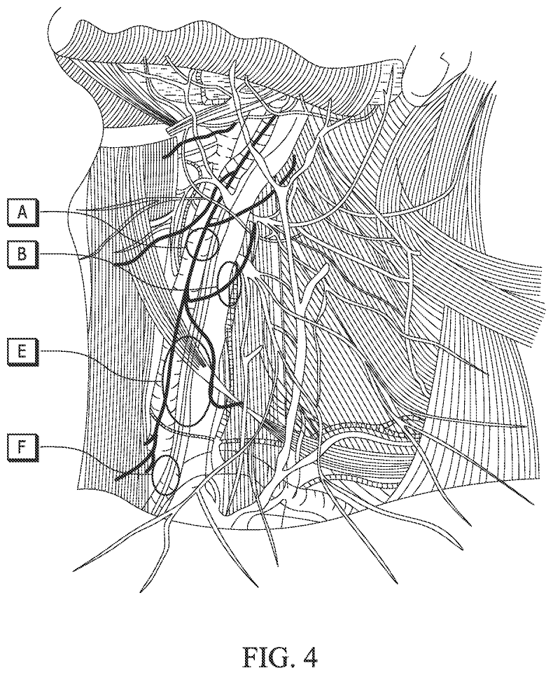

With reference to FIGS. 2-4 the infrahyoid strap muscles can be variably innervated by nerve fiber contributions from both the superior and inferior roots of the ansa cervicalis. It should be noted that FIG. 2 generally illustrates most if not all known branching patterns of the ansa cervicalis but that no actual anatomic variant with all of these branching patterns would likely exist in a single patient. Normal anatomic variants may necessitate use of one or more different target sites in different patients to achieve desired stimulation of the sternothyroid muscle 39. In certain aspects and with reference to FIG. 2, a third electrical signal is delivered to a target site proximate to the ansa cervicalis 33 that also innervates the superior belly of the sternohyoid muscle 37a and/or inferior belly of the sternohyoid muscle 37b to activate part of or all of the sternohyoid muscle 37. For example, an exemplary target site includes target site A, which can be proximate to or at the branch point 43 of the superior root of the ansa cervicalis 27 innervating the sternohyoid muscle 37 such that the sternohyoid muscle 37 is activated as well as the sternothyroid muscle 39. In certain aspects, delivering a fourth electrical signal to target site A proximate to the superior root of the ansa cervicalis 27 can also activate part or all of the omohyoid muscle 41 (a and b). If the target site were distal to the superior root of the ansa cervicalis 27 but not including branch point 1000 (e.g. placed in site G), an electrical signal may only activate the sternohyoid muscle 37 and/or omohyoid muscle 41 and not necessarily the sternothyroid muscle 39 along with the sternohyoid muscle 37 and/or omohyoid muscle 41. Without wishing to be bound by a particular mechanism of action, it is believed that activation of at least the sternothyroid muscle 39, including the sternothyroid muscle 39, the sternohyoid muscle 37, and the omohyoid muscle 41 can stiffen the patient's upper airway thereby improving the patient's SDB.

In certain aspects, an electrical signal is delivered to target site B proximate the ansa cervicalis (e.g. proximate to the inferior root of the ansa cervicalis 35) also innervating the sternothyroid muscle 39 and sternohyoid muscle 37 and omohyoid muscle 41 to activate one or more of the innervated muscles. In certain aspects, an electrical signal can be delivered simultaneously to target sites A and B proximate the ansa cervicalis 31 in order to stimulate nerve branches from both the superior root 27 and inferior root 35 of the ansa cervicalis innervating the sternothyroid muscle 39 as well as the sternohyoid muscle 37 and omohyoid muscle 41. In certain aspects, delivering an electrical signal to target site E (e.g. proximate to or at the branch point of the common trunk nerve or nerves 1000 arising from the loop of the ansa cervicalis 33 combining nerve fibers from the superior root 27 and inferior root 35 and supplying at least the sternothyroid muscle 39 and variably the sternohyoid muscle 37 and omohyoid muscle 41) can activate at least the sternothyroid muscle 39 and in certain aspects, the sternohyoid muscle 37 and in certain aspects the omohyoid muscle 41. In certain aspects, delivering an electrical signal to target site F (e.g. proximate to or at the branch point of the sternothyroid muscle nerve or nerves from the common trunk 1001) can activate the sternothyroid muscle 39. The branches to the sternothyroid muscle can be a single nerve fiber or several closely located nerve fibers traveling together. It should be noted that the above target sites are only exemplary and a therapy device, such as electrode or electrodes, can be placed at other parts of the ansa cervicalis including branches thereof. Further, stimulation can be applied to any combination of the above-described sites and branches. For example, for target site E, a therapy device, such as an electrode or electrodes, can be placed proximal or distal to the branch to the omohyoid muscle such that stimulation is capturing only the sternothyroid/sternohyoid fibers. As another example, for target site F, a cuff electrode or electrodes could surround a single fiber or multiple fibers innervating the sternothyroid muscle.

As stated above, in certain aspects, a method of improving SDB comprises stimulating the ansa cervicalis to activate one or more infrahyoid strap muscles in combination with stimulating the phrenic nerve to activate the diaphragm. Phrenic nerve stimulation can affect upper airway collapsibility. Stimulating the phrenic nerve in isolation can cause airway collapse instead of protecting against it, as diaphragm descent intrinsically generates a negative pressure gradient within the pharyngeal lumen that can cause collapse before the stabilizing effects are realized. Stimulating both the ansa cervicalis and the phrenic nerve can provide the ability to control airway collapsibility to a greater degree than either one in isolation by enabling individualized control of caudal traction and thoracic expansion through the respiratory cycle.

In certain aspects, methods of improving SDB involve stimulating additional nerves or combination of nerves in conjunction with stimulation of the ansa cervicalis and the phrenic nerve. For example, in certain aspects, a method of improving SDB comprises additionally delivering an electrical signal to a target site proximate to a hypoglossal nerve innervating the genioglossus muscle to activate the genioglossus muscle. A target site can be proximate to the hypoglossal nerve such that delivering an electrical signal activates the motor fibers of the hypoglossal nerve to activate the genioglossus muscle. In certain aspects, an electrical signal is not delivered to the hypoglossal nerve proximal to branch point 43 as it is believed that separate, therapy devices, such as electrodes, may be needed to potentially provide different strength or timing of stimulation to the ansa cervicalis and hypoglossal nerve. In other aspects, the hypoglossal nerve can be stimulated proximal or distal to the branch point of the retrusor muscle branches to the stylohyoid muscle and/or the hyoglossus muscle. Activation of the hypoglossal nerve can stiffen tongue musculature, reducing or eliminating pharyngeal obstruction generated by posterior collapse of the tongue base that would not otherwise be treated by ansa cervicalis or phrenic nerve stimulation, for example.

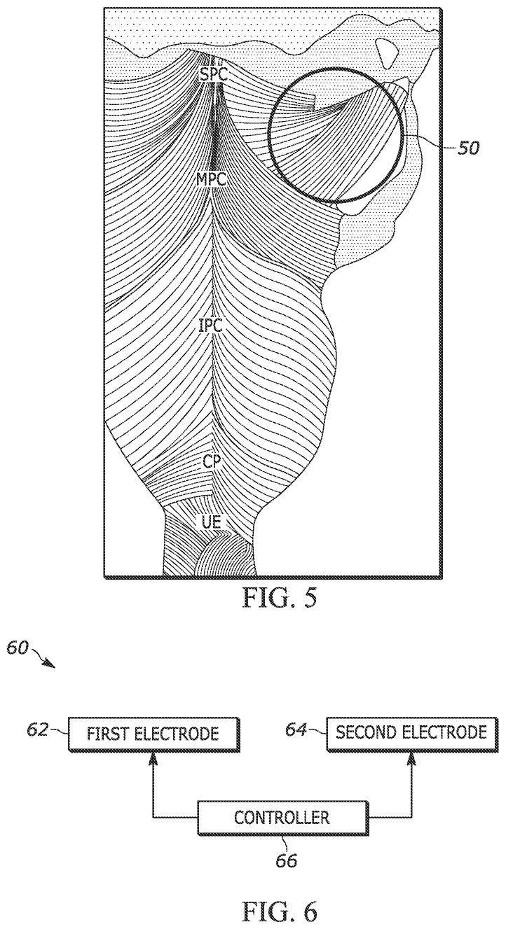

In certain aspects, a method of improving SDB comprises additionally delivering an electrical signal to an efferent fiber of the glossopharyngeal nerve innervating one or more pharyngeal constrictor muscles or a stylopharyngeus muscle to activate the one or more pharyngeal constrictor muscles or the stylopharyngeus muscle. An exemplary target site 50 is identified in FIG. 5. The target site can comprise the pharyngeal plexus or a branch thereof, for example. When activated, such one or more pharyngeal constrictor muscles can increase pharyngeal muscle tone to reduce pharyngeal airway collapsibility. When activated, a stylopharyngeus muscle can move the pharyngeal wall laterally.

The side walls of the pharynx are constructed from the pharyngeal constrictors, which are innervated by the pharyngeal plexus that include fibers from cranial nerves IX and X. The nerves that innervate these muscles form a plexus over the outside surface of the pharyngeal constrictor muscles and then penetrate the pharyngeal constrictor muscles to reach the palatoglossus and palatopharyngeus muscles. Research suggests the motor branches of cranial nerve IX may be responsible for respiratory control of the constrictor muscles and that they can be identified in the region of the stylopharyngeus muscle. Increased constrictor muscle tone during respiration can reduce pharyngeal collapsibility by stiffening the pharyngeal walls. Stiffening of the pharyngeal walls without complete constriction may render stimulation of the ansa cervicalis and hypoglossal nerve more effective. Stimulation of the stylopharyngeus muscle may increase airway caliber by moving the pharyngeal wall laterally and may also counterbalance a pharyngeal narrowing component of constrictor muscle activation that may occur if muscle activation advances beyond initial pharyngeal wall stiffening.

As stated above, stimulating an efferent fiber of the glossopharyngeal nerve innervating one or more pharyngeal constrictor muscles or a stylopharyngeus muscle can be combined with stimulation of the ansa cervicalis and the phrenic nerve. Stimulation of the ansa cervicalis may anchor the inferior end of the pharynx by preventing upward movement of the thyroid cartilage and hyoid bone, which may allow contraction of the pharyngeal constrictor muscles and palatopharyngeus muscle to work against a solid anchor as opposed to a mobile insertion point, increasing the effectiveness of co-stimulation of the ansa cervicalis with stimulation of an efferent fiber of the glossopharyngeal nerve or stimulation of the palatopharyngeus muscle.

In certain embodiments, an electrical signal is delivered to efferent fibers of the glossopharyngeal nerve independent of sensory or input signals detected or sensed regarding the neuromuscular state of the airway. For example, an electrical signal can be delivered on a tonic basis or on a duty cycle independent of sensory or input signals detected or sensed regarding the neuromuscular state of the airway. In other words, even though sensory or input signals regarding the neuromuscular state of the airway can be measured, such sensory information may not dictate the stimulation parameters of the electrical signal delivered to the target site in certain embodiments. In certain aspects, an electrical signal is not delivered to sensory fibers that innervate the mucosal layers of the pharynx wall.

In certain aspects, a method of improving SDB comprises additionally delivering an electrical signal to a nerve that innervates a palatal muscle, such as the palatoglossus muscle, the palatopharyngeus muscle, or both to improve SBD in a patient suffering therefrom. The palatoglossus muscle and palatopharyngeus muscle are muscles of the soft palate (also referred to as “palatal muscles”). The palatoglossus muscle originates from the palatine aponeurosis at the posterior part of the hard palate. It descends inferolaterally to insert into the posterolateral surface of the tongue. During its course through the posterior part of the oral cavity, it is covered medially by a mucous membrane, so forming the palatoglossus arch. The palatoglossus muscle functions to close off the oral cavity from the oropharynx by elevating the posterior tongue and drawing the soft palate inferiorly. This muscle is innervated by a branch of the pharyngeal plexus, which functions independently of the hypoglossal nerve and the rest of the intrinsic and extrinsic tongue musculature. The palatopharyngeus muscle forms the palatopharyngeal arch. It attaches superiorly to the hard palate and palatine aponeurosis and inferiorly to the lateral wall of the pharynx and the thyroid cartilage. It functions to tense the soft palate and pull the pharyngeal walls superiorly, anteriorly, and medially during swallowing, effectively closing off the nasopharynx from the oropharynx.

Such methodology of delivering an electrical signal to a nerve innervating a palatal muscle to improve SDB is different than indirect methods of muscle stimulation such as transcutaneous electrical pacing, which have been found to be inconsistent and poorly tolerated by sleeping patients. Direct intramuscular stimulation via fine wire electrode placement or other techniques would be impractical for daily use as it would require daily uncomfortable piercing of the skin or lining of the mouth to access muscle tissue. Electrical stimulation of a nerve that innervates the palatoglossus muscle and/or palatopharyngeus muscle during sleep can dilate the retropalatal space and therefore open the patient's upper airway without causing arousal from sleep. Such a method can be utilized in patients with isolated palatal collapse or in conjunction with hypoglossal nerve stimulation, ansa cervicalis stimulation and/or phrenic nerve stimulation as part of multi-level airway therapy for SBD, such as OSA.

In certain embodiments, the target site of a nerve that innervates the palatoglossus muscle and/or the palatopharyngeus muscle is the pharyngeal plexus or a branch thereof. Preferably, the electrical signal delivered to a target site of a nerve that innervates the palatoglossus muscle and/or the palatopharyngeus muscle stimulates motor fibers of the pharyngeal plexus that innervate the patient's soft palate.

In certain embodiments, a target site for stimulation is not a cranial root of an accessory nerve of the patient as targeting the cranial root of the accessory nerve or the root of the vagus nerve would result in diffuse, non-specific stimulation. For example, activation of the vagal root could simultaneously activate the levator veli palatini muscle, which opposes the action of the palatoglossus and palatopharyngeus muscles. Stimulation of the cranial root of the spinal accessory nerve could similarly cause non-specific activation of the pharyngeal plexus musculature. Moreover, the cranial root of the accessory nerve is not known to join the vagus nerve in all patients. If the cranial root of the accessory nerve did not join the pharyngeal plexus it would instead remain with the spinal root of the accessory nerve. Stimulation of the cranial root of the accessory nerve would therefore cause unintentional stimulation of the spinal root of the accessory nerve, which would cause undesirable activation of the sternocleidomastoideo trapezius muscles.

Delivering an electrical signal to any one or more of the above target sites can be accomplished by placing one or more electrodes proximate to the target site. The electrode can be placed proximate to a target site in a variety of different ways, such as, for example, transcutaneously, percutaneously, subcutaneously, intramuscularly, intraluminally, transvascularly, intravascularly, or via direct open surgical implantation.

Methods as disclosed herein can be used as part of a closed-loop system (as described in more detail below). Such a method can include sensing a physiological parameter associated with SDB, generating a sensor signal based on the physiological parameter, and activating the electrode(s) to adjust application of the electrical signal to the target site in response to the sensor signal to improve the patient's SDB.

In certain aspects and with reference to FIG. 6, a therapy delivery system to improve sleep disordered breathing is provided. Such a system 60 can comprise a first electrode 62 configured to deliver a first electrical signal to a target site proximate to an ansa cervicalis to stimulate the ansa cervicalis and activate the sternothyroid muscle. System 60 can further comprise a second electrode 64 configured to delivery a second electrical signal to a target site proximate to the phrenic nerve to stimulate the phrenic nerve and activate the diaphragm. A controller 66 can be in electrical communication with first electrode 62 and second electrode 64 and can be programmed to direct delivery of the first electrical signal to the target site to stimulate the ansa cervicalis and activate the sternothyroid muscle and can be programmed to direct delivery of the second electrical signal to the phrenic nerve to stimulate the phrenic nerve and activate the diaphragm to improve the sleep disordered breathing.

The system can include the same or different electrodes to deliver electrical signals to other target sites. For example, in certain aspects, the system can include an electrode to deliver an electrical signal to a target site proximate to the ansa cervicalis to stimulate the ansa cervicalis and activate the sternohyoid muscle; an electrode configured to deliver an electrical signal to a target site proximate to the ansa cervicalis to stimulate the ansa cervicalis and activate the omohyoid muscle; an electrode configured to deliver an electrical signal to a hypoglossal nerve to stimulate the hypoglossal nerve and activate the genioglossus muscle; an electrode configured to deliver an electrical signal to an efferent fiber of the glossopharyngeal nerve to activate one or more pharyngeal constrictor muscles or a stylopharyngeus muscle; and/or an electrode configured to deliver an electrical signal to a pharyngeal nerve plexus to activate a palatal muscle such as a palatoglossus muscle and/or a palatopharyngeus muscle. The system can include a controller programmed to direct delivery of the electrical signals to the various target sites and combination of target sites. A single controller can be in electrical communication with more than one electrode including all of the electrodes. Alternatively, each electrode can be in electrical communication with a separate controller. The electrodes can have different form factors such as, for example, an injectable microstimulator, a nerve cuff electrode, or a transcutaneous patch. Further, as stated above, multiple target sites can be stimulated by the same electrode or a single target site can be stimulated by a single electrode. The stimulation can unilateral stimulation as well as bilateral stimulation of these nerve(s).

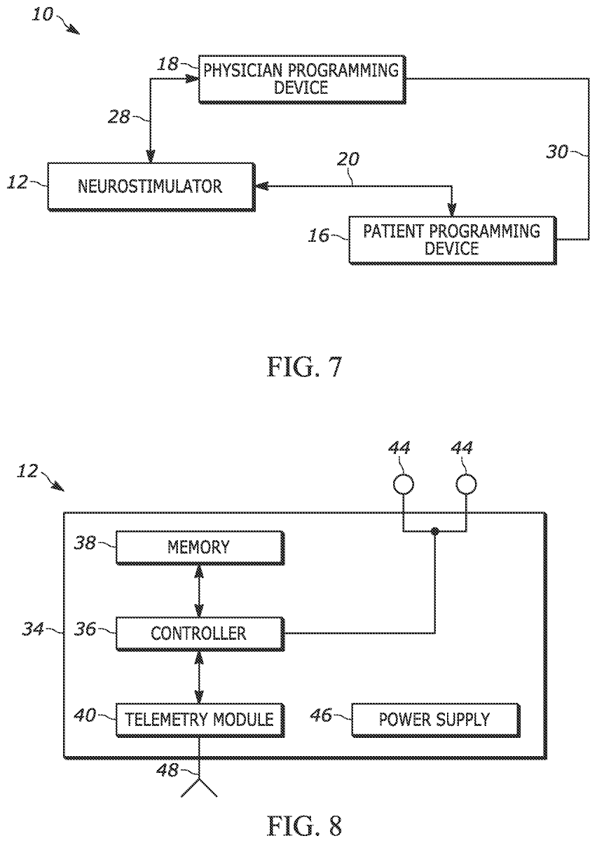

Referring to FIG. 7, in certain aspects, a neurostimulation system 10 is provided that includes a neurostimulator 12, a patient programming device 16 that bi-directionally communicates with neurostimulator 12 and a physician programming device 18. As discussed below, each component of a system can be in communication (e.g., electrical communication) with one another. In some instances, two or more components of a system can be in wireless communication with one another. In other instances, two or more components of a system can be in wired communication with one another. As such, some components of a system can be in wireless communication with one another while other components are in wired communication with one another. Further, in the illustrative embodiments disclosed herein, communication between components included in neurostimulation system 10 is configured to be bidirectional in nature. However, communication between two or more system components can be unidirectional. Further, the functionality of different components of the system can be combined into a single device. For example, the functionality of components can be combined into a single device.

In an embodiment, neurostimulator 12 includes electronic circuitry, such as one or more electronic circuits, for delivering neurostimulation pulses enclosed in a sealed housing and coupled to electrodes. In certain embodiments, neurostimulator 12 can include a primary battery cell, a rechargeable battery cell, or an inductively coupled power source for providing power for generating and delivering stimulation pulses and powering other device functions such as communication functions. Neurostimulator 12 or system 10 can include fixation members to secure the neurostimulator to tissue adjacent to the target site.

Patient programming device 16 can be a patient handheld device that is used to initiate and terminate therapy delivered by neurostimulator 12 via a bidirectional wireless telemetry link 20. Programming of neurostimulator 12 can be performed by patient programming device 16, using near- or distance-telemetry technology for establishing a bidirectional communication link 20 for transmitting data between programming device 16 and neurostimulator 12. Patient programming device 16 can be used by a patient or clinician to set a therapy protocol that is performed automatically by neurostimulator 12. Patient programming device 16 can be used to manually start and stop therapy, adjust therapy delivery parameters, and collect data from neurostimulator 12, e.g. data relating to total accumulated therapy delivery time or other data relating to device operation or measurements taken by neurostimulator 12. For example, programming device 16 can include software programmed to control one or more stimulation or control parameters associated with neurostimulator 12. Additionally, or optionally, the software comprising patient programming device 16 can be programmed to store patient therapy data, such as diary questions or physiologic measurements. Programming device 16 can also include software programmed to access remote data sources, query certain data, and then provide stimulation instructions to system 10 based on the queried data. For example, programming device 16 can include software programmed to provide neurostimulator 12 with customizable or patient-triggered alerts, e.g., indicating stimulation periods and the duration of each period, after a desired period of time (e.g., 30 minutes) after sleep onset. Programming device 16 can be embodied as a smart phone or tablet, although personal computers (PCs) may also be included.

In some embodiments, the patient can be provided with a magnet for adjusting operation of neurostimulator 12. For example, application of the magnet can turn therapy on or off or cause other binary or stepwise adjustments to neurostimulator 12 operations.

Physician programming device 18 can include increased programming and diagnostic functionality compared to patient programming device 16. For example, physician programming device 18 can be configured for programming all neurostimulation therapy control parameters, such as, but not limited to, pulse amplitude, pulse width, pulse shape, pulse frequency, duty cycle, therapy on and off times, electrode selection, and electrode polarity assignments. Patient programming device 16 can be limited to turning therapy on or off, adjusting a start time of therapy, or adjusting a pulse amplitude without giving the patient full access to full programming functions such that some programming functions and programmable therapy control parameters cannot be accessed or altered by a patient.

Physician programming device 18 can be configured to communicate directly with neurostimulator 12 via wireless, bidirectional telemetry link 28 for example during an office visit. Additionally or alternatively, physician programming device 18 can be operable as a remote programming instrument used to transmit programming commands to patient programming device 16 via a wired or wireless communication network link 30, after which patient programming device 16 automatically transmits programming data to neurostimulator 12 via bidirectional telemetry link 20 (or via wearable external device 14 and link 24). Physician programming device can be embodied as a smart phone, tablet or PC, for example.

FIG. 8 is a functional block diagram of exemplary components of neurostimulator 12 of FIG. 7 according to an embodiment of a neurostimulation system. Neurostimulator 12 can include a housing 34 enclosing a controller 36 and associated memory 38, and a telemetry module 40. Neurostimulator 12 includes a power supply 46, which can include any of a primary battery cell, a rechargeable battery cell, or a secondary coil of an externally powered system.

Controller 36 can include any one or more of a microprocessor, a digital signal processor (DSP), an application specific integrated circuit (ASIC), a field-programmable gate array (FPGA), or equivalent discrete or integrated logic circuitry. In some examples, controller 36 can include multiple components, such as any combination of one or more microprocessors, one or more controllers, one or more DSPs, one or more ASICs, or one or more FPGAs, as well as other discrete or integrated logic circuitry. The functions attributed to controller 36 herein can be embodied as software, firmware, hardware or any combination thereof. The controller can be programmed to deliver electrical signals having various characteristics. For example, the electrical signal may be constant, intermittent, varying or modulated with respect to the current, voltage, pulse-width, waveform, cycle, frequency, amplitude, and so forth. The waveform can be a sine wave, a square wave, or the like. The type of stimulation may vary and involve different waveforms. Optimal activation patterns may require a delay in one electrode before activating another or in another coordinated fashion to optimally open the airway, whether that involves simultaneous activation or staggered activation in a coordinated, adjustable fashion. The controller may be programmed to control numerous electrodes independently or in various combinations as needed to provide neuromodulation. In one example, a neurostimulation therapy protocol to improve an SDB in a patient can be stored or encoded as instructions in memory 38 that are executed by controller 36 to cause a pulse generator to deliver the therapy via electrodes 44 according to the programmed protocol. As such, a neurostimulation device can be pre-programmed with desired stimulation parameters. Although controller is illustrated in FIG. 8 as being internal to the neurostimulation device, it alternatively can be an external controller such that stimulation parameters are remotely modulated to desired settings.

Memory 38 can include computer-readable instructions that, when executed by controller 36, cause neurostimulator 12 to perform various functions attributed throughout this disclosure to the neurostimulator. The computer-readable instructions can be encoded within memory 38. Memory 38 can comprise non-transitory computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media with the sole exception being a transitory, propagating signal.

Telemetry module 40 and associated antenna 48 can be provided for establishing bidirectional communication with patient programmer 16 or physician programmer 18. Examples of communication techniques used by neurostimulator 12 and programming device 16 or 18 include low frequency or radiofrequency (RF) telemetry, which can be an RF link established via Bluetooth, WiFi, or MICS, for example. Antenna 48 can be located within, along or extend externally from housing 34.

Electrodes 44 can be located along an exterior surface of housing 44 and can be coupled to pulse generator 42 via insulated feedthroughs or other connections as will be further described below. In other embodiments, electrodes 44 can be carried by a lead or insulated tether electrically coupled to a controller via appropriate insulated feedthroughs or other electrical connections crossing sealed housing 34. In still other embodiments, electrodes 44 can be incorporated in housing 34 with externally exposed surfaces adapted to be operably positioned in proximity to a target site proximate to a nerve and electrically coupled to controller 36. The electrodes can be controllable to provide electrical signals that may be varied, for example, in voltage, frequency, amplitude, waveform, pulse-width, current, intensity, duty cycle, polarity, signal pulse intensity, signal pulse u the signal pulse amplitude, the signal pulse intensity, the signal pulse duration, and combinations thereof. The electrode can also provide both positive and negative current flow from the electrode or can be capable of stopping current flow from the electrode or changing the direction of current flow from the electrode.

As stated above, an electrode can be placed on the same or different target sites. For example, if the target sites include two separate nerves or nerve segment, a separate nerve cuff electrode can be placed on each nerve or nerve segment with each nerve cuff electrode having its own cathode and anode but connected to the same controller or separate nerve cuff electrodes connected to the same controller but one nerve cuff electrode serving as the cathode and the other serving as the anode, where the electrical field generated captures both nerves or nerve segments. In certain embodiments, an electrode or electrodes configured to stimulate a nerve or nerve segment can be combined with an electrode configured to stimulate another nerve or nerve segment. Still alternatively, an electrode or electrodes configured to stimulate a nerve or nerve segment can be part of a device separate from a device configured to stimulate another nerve or nerve segment.

Power supply 46 can be a battery or other power source. The battery can be rechargeable by inductive coupling. The power supply can be inside the neurostimulator (as depicted in FIG. 8), at a remote site in or on the patient's body, or away from the patient's body in a remote location. When located away from the body, the neurostimulation device may be powered by bringing a power source external to the patient's body into contact with the patient's skin. When neurostimulator 12 is configured as an externally powered device, the power supply can be worn by the patient during sleep to provide power needed to generate stimulation pulses or can be adjacent to the patient (e.g. such as one the patient's bed, under the patient's pillow, one the patient's nightstand, etc.). For example, the power supply can be a battery-powered device including a primary coil used to inductively transmit power to a secondary coil included in the neurostimulator. The power supply can include one or more primary or rechargeable cells and therefore can include a power adaptor and plug for re-charging in a standard 110V or 220V wall outlet, for example. In some embodiments, the functionality required for transmitting power to neurostimulator 12 when neurostimulator 12 is embodied as a rechargeable or externally powered device and for programming the neurostimulator 12 for controlling therapy delivery can be implemented in a single external device.

In another aspect, system 10 can include one or more sensors (not shown) to permit open- or closed-loop control. In an open-loop system, for example, system 10 can include one or more sensors such that a patient can manage (e.g., prophylactically) improvement of the SDB based on feedback (e.g., detected signals) from the sensor(s). Such detected signals can be indicative of the onset of the SDB, such as changes in muscle or nerve electrical activity, tongue position, oropharyngeal airflow, etc. Upon noticing the signal(s), the patient can then trigger or activate the neurostimulator 12 to prevent or mitigate the SDB.

In another aspect, system 10 can include one or more sensors to permit closed-loop control by, for example, automatically responding (e.g., by activation of the neurostimulator 12) in response to a sensed physiological parameter, or a related symptom or sign, indicative of the extent or presence of the SDB. Physiological parameters include changes in muscle or nerve electrical activity, tongue position, changes in heart rate or blood pressure, pressure changes in response to respiratory effort, oropharyngeal airflow, etc. Sensors used as part of a closed- or open-loop system can be placed at any appropriate anatomical location on a patient, including a skin surface, an oral cavity, a nasal cavity, a mucosal surface, or at a subcutaneous location.

Each of the disclosed aspects and embodiments of the present disclosure may be considered individually or in combination with other aspects, embodiments, and variations of the disclosure. Unless otherwise specified, none of the steps of the methods of the present disclosure are confined to any particular order of performance. Further, while the above is described with respect to electrical stimulation, other forms of energy could be used, such as, for example, ultrasound, magnetic, or optical energy.

1. A therapy delivery system to improve sleep disordered breathing comprising: a first electrode configured to deliver a first electrical signal to a target site proximate to the ansa cervicalis to stimulate the ansa cervicalis and activate the sternothyroid muscle; a second electrode configured to deliver a second electrical signal to a target site proximate to the phrenic nerve to stimulate the phrenic nerve and activate the diaphragm; a controller in electrical communication with the first electrode and the second electrode and programmed to direct delivery of the first electrical signal to the target site to stimulate the ansa cervicalis and activate the sternothyroid muscle and programmed to direct delivery of the second electrical signal to the phrenic nerve to stimulate the phrenic nerve and activate the diaphragm to improve the sleep disordered breathing.

2. The system of claim 1, further comprising: a third electrode configured to deliver a third electrical signal to a target site proximate to the ansa cervicalis to stimulate the ansa cervicalis and activate the sternohyoid muscle, the controller being programmed to direct delivery of the third electrical signal to the target site to stimulate the ansa cervicalis and activate the sternohyoid muscle.

3. The system of claim 1, further comprising: a fourth electrode configured to deliver a fourth electrical signal to a target site proximate to the ansa cervicalis to stimulate the ansa cervicalis and activate the omohyoid muscle, the controller being programmed to direct delivery of the fourth electrical signal to the target site to stimulate the ansa cervicalis and activate the omohyoid muscle.

4. The system of claim 2, further comprising: a fourth electrode configured to deliver a fourth electrical signal to a target site proximate to the ansa cervicalis to stimulate the ansa cervicalis and activate the omohyoid muscle, the controller being programmed to direct delivery of the fourth electrical signal to the target site to stimulate the ansa cervicalis and activate the omohyoid muscle.

5. The system of claim 1, further comprising: a fifth electrode configured to deliver a fifth electrical signal to the hypoglossal nerve to stimulate the hypoglossal nerve and activate the genioglossus muscle, the controller being programmed to direct delivery of the fifth electrical signal to the hypoglossal nerve to activate the genioglossus muscle.

6. The system of claim 1, further comprising: a sixth electrode configured to deliver a sixth electrical signal to an efferent fiber of the glossopharyngeal nerve to activate one or more pharyngeal constrictor muscles or a stylopharyngeus muscle, the controller being programmed to direct delivery of the sixth electrical signal to an efferent fiber of the glossopharyngeal nerve to activate one or more pharyngeal constrictor muscles or a stylopharyngeus muscle.

7. The system of claim 6, wherein the efferent fiber is the pharyngeal nerve plexus or branch thereof.

8. The system of claim 6, wherein the controller is programmed to avoid stimulating a cranial root of an accessory nerve of the patient.

9. The system of claim 1, further comprising: a seventh electrode configured to deliver a seventh electrical signal to a pharyngeal nerve plexus or branch thereof to activate a palatoglossus muscle, a palatopharyngeus muscle, or both, the controller being programmed to direct delivery of the seventh electrical signal to the pharyngeal nerve plexus or branch thereof to activate the palatoglossus muscle, the palatopharyneus muscle, or both.

10. The system of claim 1, wherein the first and second electrodes are each a plurality of electrodes.

11. The system of claim 1, further comprising a sensor configured to sense a physiological parameter indicative of the presence, onset, and/or severity of the sleep disordered breathing.

12. The system of claim 11, wherein the controller is programmed to activate the first electrode and/or the second electrode in response to the sensed physiological parameter to improve the sleep disordered breathing.

13. The system of claim 11, wherein the sensed physiological parameter is respiratory state.

14. The system of claim 13, wherein the respiratory state is inspiration.

15. The system of claim 13, wherein the respiratory state is expiration.

16. The system of claim 11, wherein the sensed physiological parameter is sleep/wake state, sleep stage, onset/termination of sleep of the patient, or combinations thereof.

17. The system of claim 11, wherein the sensor is configured to detect a sleep disordered event and the controller is programmed to activate the first electrode and/or the second electrode to activate or pace respiration after detection of the sleep disordered breathing event.

18. The system of claim 11, wherein the sensor is configured to detect a patient's sleep state and the controller is programmed to adjust a duty cycle of the first and/or the second electrical signal after automatic detection of the patient's sleep state.

19. The system of claim 1, wherein the controller comprises: a first controller in electrical communication with the first electrode; and a second controller in electrical communication with the second electrode.

20. The system of claim 1, further comprising a patient programming device and/or a physician programming device configured to communicate with the controller to control operation of the controller.

21. A therapy delivery system for improving obstructive sleep apnea comprising: a first electrode configured to deliver a first electrical signal to the phrenic nerve to stimulate the phrenic nerve and activate the diaphragm. a controller in electrical communication with the first electrode and programmed to direct delivery of the first electrical signal to the phrenic nerve to stimulate the phrenic nerve to activate the diaphragm to improve the obstructive sleep apnea.

22.-59. (canceled)
